(19) United States
(12) Patent Application Publication
(10) Pub. No.: US 2018/0161381 A1
(43) Pub. Date: Jun. 14, 2018

(54) NUTRITIONAL COMPOSITIONS AND METHODS FOR REDUCING THE OCCURRENCE OR SEVERITY OF VIRAL INFECTIONS, BACTERIAL INFECTIONS AND VIRAL AND BACTERIAL CO-INFECTIONS

(71) Applicant: Mead Johnson Nutrition Company, Glenview, IL (US)

(72) Inventors: Sarmauli Manurung, Nijmegen (NL); Gabriele Gross, Nijmegen (NL); Teartse Tim Lambers, Nijmegen (NL); Eric A.F. van Tol, Arnhem (NL)

(21) Appl. No.: 15/375,663

(22) Filed: Dec. 12, 2016

(51) Int. Cl. A61K 35/747; A23L 33/00; A23L 33/135

(52) U.S. Cl. CPC A61K 35/747; A23L 33/40; A23Y 2220/73; A23V 2002/00; A23L 33/135

(57) ABSTRACT

The present disclosure is directed to methods for reducing the risk of developing or reducing the severity of a viral infection, bacterial infection, or viral and bacterial co-infection in a subject comprising administering to the subject a nutritional composition comprising an effective amount of a soluble mediator preparation derived from a late-exponential growth phase of a probiotic culture, such as Lactobacillus rhamnosus GG (LGG). The present disclosure, in certain embodiments, is directed to methods for reducing inflammation in a subject with a viral infection, bacterial infection, or viral and bacterial co-infections, comprising administering to the subject a nutritional composition comprising an effective amount of a soluble mediator preparation from a late-exponential growth phase of a probiotic culture, such as LGG.

### TECHNICAL FIELD

The present disclosure relates generally to compositions and methods for reducing the occurrence and severity of viral infections, bacterial infections, and viral and bacterial co-infections. More particularly, the present disclosure relates to compositions comprising a culture supernatant from a late-exponential growth phase of a probiotic culture, and methods of administering them.

### BACKGROUND

Bacterial secondary infections or co-infections associated with cases of influenza are a leading cause of severe morbidity and mortality. (Joseph et al. (2013) INFLUENZA 7(2):105-113.) This is especially true for high-risk groups, such as infants and children. The mechanisms leading to secondary or co-infection are complex. In some cases, viruses and bacteria are transmitted simultaneously. In other cases, viral infection damages respiratory epithelial cells and hampers their repair, leading to reduced mucociliary bacterial clearance and inducing aberrant immune responses which can lead to severe inflammation, bacterial colonization and infection severity, and pneumonia.

While immunizations against certain viruses and bacteria can be effective in combating viral and/or bacterial infections, they are not available for every virus or bacterium. Even when an immunization is available, it is not always effective. Further, the rapidly changing nature of certain viruses due to viral mutagenesis can make effective vaccination difficult. Anti-viral medications can be effective against some, but not all, viruses. However, anti-viral medications must be taken within 48 hours of infection to be most effective. Antibiotics may be used to combat bacterial infections, but antibiotics can have undesirable gastrointestinal and other side effects and result in antibiotic resistance.

Accordingly, there is a need in the art for alternative therapies to combat viral infections, bacterial infections, and bacterial viral and bacterial co-infections.

### BRIEF SUMMARY

Briefly, the present disclosure is directed to methods for reducing the risk of developing or reducing the severity of a viral infection, bacterial infection, and/or viral and bacterial co-infection in a subject, comprising administering to the subject a nutritional composition comprising an effective amount of a soluble mediator preparation from a late-exponential growth phase of a probiotic culture. The present disclosure is further directed to methods for reducing inflammation in a subject with a viral infection, bacterial infection, and/or viral and bacterial co-infection comprising administering to the subject a nutritional composition comprising an effective amount of a soluble mediator preparation from a late-exponential growth phase of a probiotic culture. In certain embodiments, the method results in a reduction in a pro-inflammatory cytokine selected from the group consisting of TNFα, IL-6 and IFNβ, or a reduction in neutrophil or macrophage recruitment, or a reduction in chemoattractant protein MCP-1, or an increase in IL-10.

In certain embodiments, the probiotic is Lactobacillus rhamnosus GG (LGG).

In certain embodiments, the infection is from a bacteria selected from the group consisting of Streptococcus pneumoniae, Haemophilus influenzae; Chlamydophila pneumoniae, Mycoplasma pneumoniae, Staphylococcus aureus, Moraxella catarrhalis, Legionella pneumophila, Gram-negative bacilli, Mycobacterium tuberculosis, Bordetella pertussis, Bordetella bronchiseptica, Streptococcus pyogenes, and Pseudomonas aeruginosa. In certain embodiments, the infection is from a virus selected from the group consisting of influenza A, influenza B, parainfluenza (PIV), human rhinovirus, adenovirus, respiratory syncytial virus (RSV), hantavirus, human metapneumovirus (hMPV), Coronavirus, and nontypeable H. influenza (NTHi).

In certain embodiments, the subject is an adult. In certain embodiments, the subject is a pediatric subject, such as a child, an infant or a premature infant.

In certain embodiments, the soluble mediator preparation is produced by (a) subjecting LGG to cultivation in a suitable culture medium; (b) harvesting a culture supernatant at a late exponential growth phase of the cultivation step; (c) optionally removing low molecular weight constituents from the supernatant so as to retain molecular weight constituents above 5 or 6 kDa; (d) removing any remaining cells by 0.2 μm sterile filtration to provide the soluble mediator preparation; (e) removing liquid contents from the soluble mediator preparation. Step (b) may further include removal of bacterial cells by sterile filtration.

In certain embodiments, the cultivation is batch cultivation and the late exponential phase is defined with reference to the second half of the time between the lag phase and the stationary phase of the batch-cultivation process. In certain embodiments, the late exponential phase is defined with reference to the latter quarter portion of the time between the lag phase and the stationary phase of the batch-cultivation process.

In certain embodiments, the cultivation is conducted in a culture medium devoid of polysorbates. In certain embodiments, the cultivation is conducted at a pH of from 5-7.

The effective amount can be equivalent to the amount of soluble mediators produced by about 1×10⁴ to about 1×10¹² colony forming units (cfu) of live probiotic bacteria per kg body weight per day.

In certain embodiments, the nutritional composition is a pediatric nutritional composition.

It is to be understood that both the foregoing general description and the following detailed description present embodiments of the disclosure and are intended to provide an overview or framework for understanding the nature and character of the disclosure as it is claimed. The description serves to explain the principles and operations of the claimed subject matter. Other and further features and advantages of the present disclosure will be readily apparent to those skilled in the art upon a reading of the following disclosure.

### BRIEF DESCRIPTION OF THE DRAWINGS

FIG. 1 shows weight loss over time after S. pneumoniae superinfection. LEGa refers to soluble mediator desalted by column chromatography and LEGb refers to soluble mediator desalted by ultrafiltration. Mice receiving LEGb showed a trend of reduced weight loss at 24 h and significantly less weight loss at 72 h (p=0.019) when compared to animals receiving water.

FIG. 2 shows the bacterial (S. pneumoniae) count in the (A) nasal lavage and (B) lung homogenates at 24 h or 72 h post bacterial infection. Mice receiving LEGb showed significantly less bacteria in the nose at 24 h (p=0.0079) when compared to animals receiving water

FIG. 3 shows levels of pro-inflammatory cytokines (TNFα; IL6; IFNβ) and a chemokine (MCP-1) in lung homogenates of mice 24 h post bacterial infection. Mice receiving LEGb showed a significant amount of TNFα, IL6, IFNβ and MCP-1 in the lung homogenates at 24 h when compared to animals receiving water.

FIG. 4 shows the levels of anti-inflammatory cytokine (IL10) in lung homogenates of mice 24 h and 72 h post bacterial infection. Mice receiving LEGa or LEGb maintained a significantly higher amount of IL10 in the lung homogenates at 72 h when compared to animals receiving water.

### DETAILED DESCRIPTION

Reference now will be made in detail to the embodiments of the present disclosure, one or more examples of which are set forth herein below. Each example is provided by way of explanation of the nutritional composition of the present disclosure and is not a limitation. In fact, it will be apparent to those skilled in the art that various modifications and variations can be made to the teachings of the present disclosure without departing from the scope of the disclosure. For instance, features illustrated or described as part of one embodiment, can be used with another embodiment to yield a still further embodiment.

Thus, it is intended that the present disclosure covers such modifications and variations as come within the scope of the appended claims and their equivalents. Other objects, features and aspects of the present disclosure are disclosed in or are obvious from the following detailed description. It is to be understood by one of ordinary skill in the art that the present discussion is a description of exemplary embodiments only and is not intended as limiting the broader aspects of the present disclosure.

Briefly, the present disclosure is directed to methods for reducing the risk of developing a viral infection, bacterial infection, and/or viral and bacterial co-infection or reducing the severity of a viral infection, bacterial infection, and/or viral and bacterial co-infection in a subject comprising administering to the subject a nutritional composition comprising an effective amount of a soluble mediator preparation from a late-exponential growth phase of a probiotic culture.

The present disclosure is further directed to methods for reducing inflammation in a subject with a viral infection, bacterial infection, and/or viral and bacterial co-infection comprising administering to the subject a nutritional composition comprising an effective amount of a soluble mediator preparation from a late-exponential growth phase of a probiotic culture.

“Nutritional composition” means a substance or formulation that satisfies at least a portion of a subject's nutrient requirements. The terms “nutritional(s)”, “nutritional formula(s)”, “enteral nutritional(s)”, and “nutritional supplement(s)” are used as non-limiting examples of nutritional composition(s) throughout the present disclosure. Moreover, “nutritional composition(s)” may refer to liquids, powders, gels, pastes, solids, concentrates, suspensions, or ready-to-use forms of enteral formulas, oral formulas, formulas for infants, formulas for pediatric subjects, formulas for children, growing-up milks and/or formulas for adults.

The term “enteral” means deliverable through or within the gastrointestinal, or digestive tract. “Enteral administration” includes oral feeding, intragastric feeding, transpyloric administration, or any other administration into the digestive tract. “Administration” is broader than “enteral administration” and includes parenteral administration or any other route of administration by which a substance is taken into a subject's body.

“Pediatric subject” means a human no greater than 13 years of age. In some embodiments, a pediatric subject refers to a human subject that is between birth and 8 years old. In other embodiments, a pediatric subject refers to a human subject between 1 and 6 years of age. In still further embodiments, a pediatric subject refers to a human subject between 6 and 12 years of age. The term “pediatric subject” may refer to infants (preterm or full term) and/or children, as described below.

“Infant” means a human subject ranging in age from birth to not more than one year and includes infants from 0 to 12 months corrected age. The phrase “corrected age” means an infant's chronological age minus the amount of time that the infant was born premature. Therefore, the corrected age is the age of the infant if it had been carried to full term. The term infant includes low birth weight infants, very low birth weight infants, extremely low birth weight infants and preterm infants. “Preterm” means an infant born before the end of the 37^{th} week of gestation. “Late preterm” means an infant form between the 34^{th} week and the 36^{th} week of gestation. “Full term” means an infant born after the end of the 37^{th} week of gestation. “Low birth weight infant” means an infant born weighing less than 2500 grams (approximately 5 lbs, 8 ounces). “Very low birth weight infant” means an infant born weighing less than 1500 grams (approximately 3 lbs, 4 ounces). “Extremely low birth weight infant” means an infant born weighing less than 1000 grams (approximately 2 lbs, 3 ounces).

“Child” means a subject ranging in age from 12 months to 13 years. In some embodiments, a child is a subject between the ages of 1 and 12 years old. In other embodiments, the terms “children” or “child” refer to subjects that are between one and about six years old, or between about seven and about 12 years old. In other embodiments, the terms “children” or “child” refer to any range of ages between 12 months and about 13 years.

“Children's nutritional product” refers to a composition that satisfies at least a portion of the nutrient requirements of a child. A growing-up milk is an example of a children's nutritional product.

The term “degree of hydrolysis” refers to the extent to which peptide bonds are broken by a hydrolysis method.

The term “partially hydrolyzed” means having a degree of hydrolysis which is greater than 0% but less than about 50%.

The term “extensively hydrolyzed” means having a degree of hydrolysis which is greater than or equal to about 50%.

“Infant formula” means a composition that satisfies at least a portion of the nutrient requirements of an infant. In the United States, the content of an infant formula is dictated by the federal regulations set forth at 21 C.F.R. Sections 100, 106, and 107. These regulations define macronutrient, vitamin, mineral, and other ingredient levels in an effort to simulate the nutritional and other properties of human breast milk.

The term “growing-up milk” refers to a broad category of nutritional compositions intended to be used as a part of a diverse diet in order to support the normal growth and development of a child between the ages of about 1 and about 6 years of age.

“Milk-based” means comprising at least one component that has been drawn or extracted from the mammary gland of a mammal. In some embodiments, a milk-based nutritional composition comprises components of milk that are derived from domesticated ungulates, ruminants or other mammals or any combination thereof. Moreover, in some embodiments, milk-based means comprising bovine casein, whey, lactose, or any combination thereof. Further, “milk-based nutritional composition” may refer to any composition comprising any milk-derived or milk-based product known in the art.

“Nutritionally complete” means a composition that may be used as the sole source of nutrition, which would supply essentially all of the required daily amounts of vitamins, minerals, and/or trace elements in combination with proteins, carbohydrates, and lipids. Indeed, “nutritionally complete” describes a nutritional composition that provides adequate amounts of carbohydrates, lipids, essential fatty acids, proteins, essential amino acids, conditionally essential amino acids, vitamins, minerals and energy required to support normal growth and development of a subject.

Therefore, a nutritional composition that is “nutritionally complete” for a preterm infant will, by definition, provide qualitatively and quantitatively adequate amounts of carbohydrates, lipids, essential fatty acids, proteins, essential amino acids, conditionally essential amino acids, vitamins, minerals, and energy required for growth of the preterm infant.

A nutritional composition that is “nutritionally complete” for a full term infant will, by definition, provide qualitatively and quantitatively adequate amounts of all carbohydrates, lipids, essential fatty acids, proteins, essential amino acids, conditionally essential amino acids, vitamins, minerals, and energy required for growth of the full term infant.

A nutritional composition that is “nutritionally complete” for a child will, by definition, provide qualitatively and quantitatively adequate amounts of all carbohydrates, lipids, essential fatty acids, proteins, essential amino acids, conditionally essential amino acids, vitamins, minerals, and energy required for growth of a child.

As applied to nutrients, the term “essential” refers to any nutrient that cannot be synthesized by the body in amounts sufficient for normal growth and to maintain health and that, therefore, must be supplied by the diet. The term “conditionally essential” as applied to nutrients means that the nutrient must be supplied by the diet under conditions when adequate amounts of the precursor compound is unavailable to the body for endogenous synthesis to occur.

“Prebiotic” means a non-digestible food ingredient that beneficially affects the host by selectively stimulating the growth and/or activity of one or a limited number of bacteria in the digestive tract that can improve the health of the host.

As used herein, “lactoferrin from a non-human source” means lactoferrin which is produced by or obtained from a source other than human breast milk. For example, lactoferrin for use in the present disclosure includes human lactoferrin produced by a genetically modified organism as well as non-human lactoferrin. The term “organism”, as used herein, refers to any contiguous living system, such as animal, plant, fungus or micro-organism. Exemplary non-human sourced lactoferrin includes bovine lactoferrin.

As used herein, “non-human lactoferrin” means lactoferrin that has an amino acid sequence that is different than the amino acid sequence of human lactoferrin.

All percentages, parts and ratios as used herein are by weight of the total formulation, unless otherwise specified.

All amounts specified as administered “per day” may be delivered in one unit dose, in a single serving or in two or more doses or servings administered over the course of a 24 hour period.

The nutritional compositions of the present disclosure may be substantially free of any optional or selected ingredients described herein, provided that the remaining nutritional composition still contains all of the required ingredients or features described herein. In this context, and unless otherwise specified, the term “substantially free” means that the selected composition may contain less than a functional amount of the optional ingredient, typically less than 0.1% by weight, and also, including zero percent by weight of such optional or selected ingredient.

All references to singular characteristics or limitations of the present disclosure shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The methods and compositions of the present disclosure, including components thereof, can comprise, consist of, or consist essentially of the essential elements and limitations of the embodiments described herein, as well as any additional or optional ingredients, components or limitations described herein or otherwise useful in nutritional compositions.

As used herein, the term “about” should be construed to refer to both of the numbers specified as the endpoint(s) of any range. Any reference to a range should be considered as providing support for any subset within that range.

“Probiotic” means a microorganism with low or no pathogenicity that exerts a beneficial effect on the health of the host. The term “inactivated probiotic” means a probiotic wherein the metabolic activity or reproductive ability of the referenced probiotic has been reduced or destroyed. The “inactivated probiotic” does, however, still retain, at the cellular level, its cell structure or other structure associated with the cell, for example exopolysaccharide and at least a portion of its biological glycol-protein and DNA/RNA structure. As used herein, the term “inactivated” is synonymous with “non-viable”.

Any probiotic known in the art may be used. In a particular embodiment, the probiotic may be selected from any Lactobacillus species, Lactobacillus rhamnosus GG (LGG) (ATCC number 53103), Bifidobacterium species, Bifidobacterium longum AH1206 (NCIMB: 41382), Bifidobacterium breve AH1205 (NCIMB: 41387), Bifidobacterium infantis 35624 (NCIMB: 41003), and Bifidobacterium animalis subsp. lactis BB-12 (DSM No. 10140) or any combination thereof.

In particular embodiments, the probiotic is Lactobacillus rhamnosus GG (Lactobacillus G.G., strain ATCC 53103). LGG is a bacterium that has been isolated from a fecal sample of a healthy human subject. It is widely recognized as a probiotic. It was disclosed in U.S. Pat. No. 5,032,399 to Gorbach, et al., which is herein incorporated in its entirety, by reference thereto. LGG is not resistant to most antibiotics, stable in the presence of acid and bile, and attaches avidly to mucosal cells of the human intestinal tract. It persists for 1-3 days in most individuals and up to 7 days in 30% of subjects. In addition to its colonization ability, LGG also beneficially affects mucosal immune responses. LGG is deposited with the depository authority American Type Culture Collection under accession number ATCC 53103. While not wishing to be bound by theory, it is believed that a nutritional composition comprising supernatant from a probiotic culture, and in particular embodiments, LGG, reduces the risk of developing a viral infection, bacterial infection, and/or viral and bacterial co-infection and/or reduces the severity of a viral infection, bacterial infection, and/or viral and bacterial co-infection. Exemplary bacterial infections treatable according to the methods disclosed herein include Streptococcus pneumoniae, Haemophilus influenzae; Chlamydophila pneumoniae, Mycoplasma pneumoniae, Staphylococcus aureus, Moraxella catarrhalis, Legionella pneumophila, Gram-negative bacilli, Mycobacterium tuberculosis, Bordetella pertussis, Bordetella bronchiseptica, Streptococcus pyogenes, and Pseudomonas aeruginosa.

Exemplary viral infections treatable according to the methods disclosed herein include influenza A, influenza B, parainfluenza, human rhinovirus, adenovirus, respiratory syncytial virus (RSV), hantavirus, human metapneumovirus, Coronavirus, and nontypeable H. influenza (NTHi).

As disclosed herein, a culture supernatant from a probiotic culture can reduce inflammation (e.g., lung inflammation) when administered to a subject in need thereof, i.e., a subject having a viral infection, bacterial infection, and/or viral and bacterial co-infection. A reduction in inflammation can be determined by any means known in the art, including, for example, by measuring a reduction in pro-inflammatory cytokines (e.g., IL-6 and IFNβ) or a chemoattractant protein (e.g., MCP-1), and/or by an increase in an anti-inflammatory cytokine (e.g., IL-10), as compared to a subject with a co-infection to whom a culture supernatant from a probiotic culture has not been administered. It is further believed that the preventative and therapeutic benefits can be attributed to the mixture of components (including proteinaceous materials, and possibly including (exo)polysaccharide materials) that are released into the culture medium at a late stage of the exponential (or “log”) phase of cultivation of LGG. The composition will be hereinafter referred to as “soluble mediator preparation.”

A soluble mediator preparation of the present disclosure can be prepared as described below. Furthermore, preparation of an LGG soluble mediator preparation is described in US 20130251829 and US 20110217402, each of which is incorporated by reference in its entirety. The stages recognized in batch and fed-batch (or semi-batch) cultivation of bacteria are known to the skilled person. These are the “lag,” the “log” (“logarithmic” or “exponential”), the “stationary” and the “death” (or “logarithmic decline”) phases. In all phases during which live bacteria are present, the bacteria metabolize nutrients from the media, and secrete (exert, release) materials into the culture medium. The composition of the secreted material at a given point in time of the growth stages is not generally predictable.

The present disclosure further relates to processes for preparing a probiotic soluble mediator preparation. In a preferred embodiment, a composition according to the disclosure and/or embodiments thereof is obtainable by a process comprising the steps of (a) subjecting a probiotic such as LGG to cultivation in a suitable culture medium using a batch or fed-batch (semi-batch) process; (b) harvesting a culture supernatant at a late exponential growth phase of the cultivation step, which phase is defined with reference to the second half of the time between the lag phase and the stationary phase of the batch- or fed-batch (semi-batch)-cultivation process; (c) optionally removing low molecular weight constituents from the supernatant so as to retain molecular weight constituents above 5-6 kiloDaltons (kDa); (d) removal of any remaining cells using 0.22 μm sterile filtration to provide the soluble mediator preparation; (e) removing liquid contents from the soluble mediator preparation.

In certain embodiments, secreted materials are harvested from a late exponential phase. The late exponential phase occurs in time after the mid exponential phase (which is halftime of the duration of the exponential phase, hence the reference to the late exponential phase as being the second half of the time between the lag phase and the stationary phase). In certain embodiments, the term “late exponential phase” refers to the latter quarter portion of the time between the lag phase and the stationary phase of the LGG batch- or fed-batch (semi-batch)-cultivation process. In a preferred embodiment of the present disclosure and embodiments thereof, harvesting of the culture supernatant is at a point in time of 75% to 85% of the duration of the exponential phase, and most preferably is at about 5/6 of the time elapsed in the exponential phase.

In certain embodiments, “late exponential phase” refers to the time at which the glucose concentration in the media drops below 1%.

In certain embodiments, supernatant from a continuous bacterial culture is used for producing the soluble mediator preparation as described herein (e.g., the supernatant is harvested from the culture and can be filtered, e.g., by ultrafiltration or column chromatography to remove lower molecular weight components).

The term “cultivation” or “culturing” refers to the propagation of microorganisms, in this case LGG, on or in a suitable medium. Such a culture medium can be of a variety of kinds, and is particularly a liquid broth, as is customary in the art. A preferred broth, e.g., is MRS broth as generally used for the cultivation of lactobacilli. MRS broth generally comprises polysorbate, acetate, magnesium and manganese, which are known to act as special growth factors for lactobacilli, as well as a rich nutrient base. One exemplary culture medium comprises (amounts in g/liter): peptone from casein 10.0; yeast extract 4.0; D(+)-glucose 20.0; dipotassium hydrogen phosphate 2.0; Tween® 80 1.0; triammonium citrate 2.0; sodium acetate 5.0; magnesium sulfate 0.2; manganese sulfate 0.04. Another exemplary culture medium comprises glucose H₂O 66 g/kg, demineralized water 84 g/kg, Na-acetate 3H₂O 10 g/kg, NH₄Cl 2.6 g/kg, Na₃-citrate 2H₂O 4.8 g/kg, K₂HPO₄ 4.0 g/kg, MgSO₄ 7H₂O 0.4 g/kg, MnSO₄H2O 0.08 g/kg, yeast extract 46 g/kg, and demineralized water 782 g/kg.

In certain embodiments, the culture supernatant preparation is incorporated into an infant formula or other nutritional composition. The harvesting of secreted bacterial products brings about a problem that the culture media cannot easily be deprived of undesired components. This specifically relates to nutritional products for relatively vulnerable subjects, such as infant formula or clinical nutrition. This problem is not incurred if specific components from a culture supernatant are first isolated, purified, and then applied in a nutritional product. However, it is desired to make use of a more complete culture supernatant. This would serve to provide a composition better reflecting the natural action of the probiotic (e.g. LGG) in the context of a nutritional supplementation.

Accordingly, it is desired to ensure that the composition harvested from LGG cultivation does not contain components (as may present in the culture medium) that are not desired, or generally accepted, in such formula. With reference to polysorbate regularly present in MRS broth, media for the culturing of bacteria may include an emulsifying non-ionic surfactant, e.g. on the basis of polyethoxylated sorbitan and oleic acid (typically available as Tween® polysorbates, such as Tween® 80). Whilst these surfactants are frequently found in food products, e.g. ice cream, and are generally recognized as safe, they are not in all jurisdictions considered desirable, or even acceptable for use in nutritional products for relatively vulnerable subjects, such as infant formula or clinical nutrition.

Therefore, in some embodiments, a preferred culture medium of the disclosure is devoid of polysorbates such as Tween 80. In a preferred embodiment of the disclosure and/or embodiments thereof the culture medium may comprise an oily ingredient selected from the group consisting of oleic acid, linseed oil, olive oil, rape seed oil, sunflower oil and mixtures thereof. It will be understood that the full benefit of the oily ingredient is attained if the presence of a polysorbate surfactant is essentially or entirely avoided.

More particularly, in certain embodiments, an MRS medium is devoid of polysorbates. Also preferably the medium comprises, optionally one or more of the foregoing oils, peptone (typically 0-10 g/L, especially 0.1-10 g/L), yeast extract (typically 4-50 g/L), D(+) glucose (typically 20-70 g/L), dipotassium hydrogen phosphate (typically 2-4 g/L), sodium acetate trihydrate (typically 4-5 g/L), triammonium citrate (typically 2-4 g/L), magnesium sulfate heptahydrate (typically 0.2-0.4 g/L) and/or manganese sulfate tetrahydrate (typically 0.05-0.08 g/L).

The culturing is generally performed at a temperature of 20° C. to 45° C., more particularly at 35° C. to 40° C., and more particularly at 37° C. In some embodiments, the culture has a neutral pH, such as a pH of between pH 5 and pH 7, preferably pH 6. In some embodiments, the final soluble mediator composition has a neutral pH, such as a pH of between pH 5 and pH 7, preferably pH 6.

In some embodiments, the time point during cultivation for harvesting the culture supernatant, i.e., in the aforementioned late exponential phase, can be determined, e.g. based on the OD600 nm and glucose concentration. OD600 refers to the optical density at 600 nm, which is a known density measurement that directly correlates with the bacterial concentration in the culture medium.

The culture supernatant can be harvested by any known technique for the separation of culture supernatant from a bacterial culture. Such techniques are known in the art and include, e.g., centrifugation, filtration, sedimentation, and the like. In some embodiments, LGG cells are removed from the culture supernatant using 0.22 μm sterile filtration. The probiotic soluble mediator preparation thus obtained may be used immediately, or be stored for future use. In the latter case, the preparation will generally be refrigerated, frozen or lyophilized. The preparation may be concentrated or diluted, as desired.

The soluble mediator preparation is believed to comprise a mixture of amino acids, oligo- and polypeptides, and proteins, of various molecular weights. The composition is further believed to comprise polysaccharide structures and/or nucleotides.

In some embodiments, the soluble mediator preparation of the present disclosure excludes lower molecular weight components, generally below 6 kDa, or even below 5 kDa. In these and other embodiments, the soluble mediator preparation does not include lactic acid and/or lactate salts. These lower molecular weight components can be removed, for example, by filtration (e.g., ultrafiltration) or column chromatography. In some embodiments, the culture supernatant is subjected to ultrafiltration with a 5 kDa membrane in order to retain constituents over 5 kDa. In other embodiments, the culture supernatant is desalted using column chromatography to retain constituents over 6 kDa.

The soluble mediator preparation of the present disclosure can be formulated in various ways for administration to subjects (e.g., pediatric subjects). For example, the soluble mediator preparation can be used as such, e.g. incorporated into capsules for oral administration, or in a liquid nutritional composition such as a drink (e.g., a ready-to-drink infant formula), or it can be processed before further use. Such processing generally involves separating the compounds from the generally liquid continuous phase of the supernatant. This preferably is done by a drying method, such as spray-drying or freeze-drying (lyophilization). In a preferred embodiment of the spray-drying method, a carrier material will be added before spray-drying, e.g., maltodextrin DE29. In certain embodiments, the soluble mediator preparation is incorporated into a powdered infant formula.

Nutritional compositions comprising a probiotic bacteria soluble mediator preparation, such as the LGG soluble mediator preparation of the present disclosure, advantageously possess preventative and therapeutic activities with respect to viral infections, bacterial infections, and/or viral and bacterial co-infections.

The present nutritional compositions comprising a LGG soluble mediator preparation may accordingly be particularly useful in treating subjects, particularly pediatric subjects, co-infected with bacteria and a virus. In certain embodiments, the infection is a respiratory infection, such as influenza and/or pneumonia. In certain embodiments, the co-infection comprises influenza A virus and S. pneumoniae. In certain embodiments, the co-infection comprises Influenza A virus (IAV) and Staphylococcus aureus. In certain embodiments, the co-infection comprises respiratory syncytial virus (RSV) and Strep. Pneumoniae. In certain embodiments, the co-infection comprises RSV and Haemophilus influenzae

In certain embodiments, a nutritional composition comprising an LGG soluble mediator preparation as described herein can effectively prevent co-infection in a subject who is already infected with a single viral or bacterial strain (e.g., a viral or bacterial strain which causes respiratory infection). In certain embodiments, a nutritional composition comprising an LGG soluble mediator preparation as described herein can effectively prevent simultaneous viral and bacterial co-infection in a subject who does not already have an infection.

In certain embodiments, a nutritional composition comprising a soluble mediator preparation as described herein can effectively treat a viral infection, bacterial infection, and/or viral and bacterial co-infection. Effective treatment can include a reduction in one or more symptoms of the infection or co-infection, including, a reduction in viral load and/or bacterial count, a reduced need for respiratory assistance (e.g., ventilator, supplementary oxygen, etc.), reduced hospital stay, reduced absence (i.e., fewer days absent) from day care because of illness, reduced likelihood of developing complications, reduction in antibiotic treatments for respiratory infection, etc.

A nutritional composition comprising a probiotic bacteria soluble mediator preparation, such as the LGG soluble mediator preparation of the present disclosure, also advantageously reduces inflammation in subjects, particularly pediatric subjects co-infected with bacteria and a virus. In certain embodiments, the inflammation is lung inflammation. In other embodiments, the inflammation is middle ear inflammation, inducing Acute Otitis Media (AOM).

Reduction in inflammation can be determined by measuring a reduction in a pro-inflammatory cytokine such as IL-6, IFNβ, IL-β, TNF alpha or neutrophil and macrophage recruitment or a chemoattractant protein such as MCP-1 or by measuring an increase in an anti-inflammatory cytokine such as IL-10. Methods for measuring levels of pro-inflammatory cytokines, chemoattractant proteins and anti-inflammatory cytokines are well known in the art and include antibody-based assays (e.g., ELISA) complemented with mRNA determination.

In order for the soluble mediator preparation of the disclosure (e.g., the soluble mediator preparation incorporated into a nutritional composition) to exert its beneficial effect or effects, it is to be ingested by a subject in need thereof. The form of administration of the soluble mediator preparation is not critical. In some embodiments, the composition is administered to a subject via tablets, pills, encapsulations, caplets, gel caps, capsules, oil drops, or sachets. In another embodiment, the composition is encapsulated in a sugar, fat, or polysaccharide.

In other embodiments, the soluble mediator preparation is incorporated into a nutritional composition, such as a children's nutritional product such as a follow-on formula, growing up milk, beverage, milk, yogurt, fruit juice, fruit-based drink, chewable tablet, cookie, cracker, or a milk powder. In other embodiments, the product may be an infant's nutritional product, such as an infant formula or a human milk fortifier. When the soluble mediator preparation is incorporated into a nutritional composition, in certain embodiments, it is spray dried or freeze dried prior to incorporation.

The LGG soluble mediator preparation of the present disclosure, whether added in a separate dosage form or via a nutritional product, will generally be administered in an amount effective to reduce the risk of developing a viral and bacterial co-infection or to reduce the severity of a viral and bacterial co-infection. The effective amount is preferably equivalent to 1×10⁴ to about 1×10¹² cell equivalents of live probiotic bacteria per kg body weight per day, and more preferably 10⁸-10⁹ cell equivalents per kg body weight per day. In other embodiments, the amount of cell equivalents may vary from about 1×10⁴ to about 1.5×10¹⁰ cell equivalents of probiotic(s) per 100 Kcal. In some embodiments the amount of probiotic cell equivalents may be from about 1×10⁶ to about 1×10⁹ cell equivalents of probiotic(s) per 100 Kcal nutritional composition. In certain other embodiments the amount of probiotic cell equivalents may vary from about 1×10⁷ to about 1×10⁸ cell equivalents of probiotic(s) per 100 Kcal of nutritional composition. Cell equivalent is based on the number of LGG cells at the endpoint of the LGG cultivation time before separating the supernatant for further processing.

The present LGG soluble mediator preparation may also be administered with lactoferrin. Lactoferrins are single chain polypeptides of about 80 kD containing 1-4 glycans, depending on the species. The 3-D structures of lactoferrin of different species are very similar, but not identical. Each lactoferrin comprises two homologous lobes, called the N- and C-lobes, referring to the N-terminal and C-terminal part of the molecule, respectively. Each lobe further consists of two sub-lobes or domains, which form a cleft where the ferric ion (Fe³⁺) is tightly bound in synergistic cooperation with a (bi)carbonate anion. These domains are called N1, N2, C1 and C2, respectively. The N-terminus of lactoferrin has strong cationic peptide regions that are responsible for a number of important binding characteristics. Lactoferrin has a very high isoelectric point (˜pl 9) and its cationic nature plays a major role in its ability to defend against bacterial, viral, and fungal pathogens. There are several clusters of cationic amino acids residues within the N-terminal region of lactoferrin mediating the biological activities of lactoferrin against a wide range of microorganisms. For instance, the N-terminal residues 1-47 of human lactoferrin (1-48 of bovine lactoferrin) are critical to the iron-independent biological activities of lactoferrin. In human lactoferrin, residues 2 to 5 (RRRR) and 28 to 31 (RKVR) are arginine-rich cationic domains in the N-terminus especially critical to the antimicrobial activities of lactoferrin. A similar region in the N-terminus is found in bovine lactoferrin (residues 17 to 42; FKCRRWQWRMKKLGAPSITCVRRAFA).

As described in “Perspectives on Interactions Between Lactoferrin and Bacteria” (BIOCHEMISTRY AND CELL BIOLOGY, pp 275-281 (2006)), lactoferrins from different host species may vary in their amino acid sequences though commonly possess a relatively high isoelectric point with positively charged amino acids at the end terminal region of the internal lobe. Suitable non-human lactoferrins for use in the present disclosure include, but are not limited to, those having at least 48% homology with the amino acid sequence of human lactoferrin. For instance, bovine lactoferrin (“bLF”) has an amino acid composition which has about 70% sequence homology to that of human lactoferrin. In some embodiments, the non-human lactoferrin has at least 55% homology with human lactoferrin and in some embodiments, at least 65% homology. Non-human lactoferrins acceptable for use in the present disclosure include, without limitation, bLF, porcine lactoferrin, equine lactoferrin, buffalo lactoferrin, goat lactoferrin, murine lactoferrin and camel lactoferrin. In particular embodiments, the lactoferrin is bLF.

In one embodiment, lactoferrin is present in the nutritional composition in an amount ranging from about 10 mg/100 Kcal to about 200 mg/100 Kcal. In certain embodiments, the lactoferrin is present in an amount ranging from about 15 mg/100 Kcal to about 100 mg/150 Kcal. In still another embodiment, particularly where the nutritional composition is an infant formula, the lactoferrin is present in the nutritional composition in an amount ranging from about 60 mg/100 Kcal to about 150 mg/100 Kcal or about 60 mg/100 Kcal to about 100 mg/100 Kcal.

The bLF that is used in certain embodiments may be any bLF isolated from whole milk and/or having a low somatic cell count, wherein “low somatic cell count” refers to a somatic cell count less than 200,000 cells/mL. By way of example, suitable bLF is available from Tatua Co-operative Dairy Co. Ltd., in Morrinsville, New Zealand, from FrieslandCampina Domo in Amersfoort, Netherlands or from Fonterra Co-Operative Group Limited in Auckland, New Zealand.

Lactoferrin for use in the present disclosure may be, for example, isolated from the milk of a non-human animal or produced by a genetically modified organism. For example, in U.S. Pat. No. 4,791,193, incorporated by reference herein in its entirety, Okonogi et al. discloses a process for producing bovine lactoferrin in high purity. Generally, the process as disclosed includes three steps. Raw milk material is first contacted with a weakly acidic cationic exchanger to absorb lactoferrin followed by the second step where washing takes place to remove nonabsorbed substances. A desorbing step follows where lactoferrin is removed to produce purified bovine lactoferrin. Other methods may include steps as described in U.S. Pat. Nos. 7,368,141, 5,849,885, 5,919,913 and 5,861,491, the disclosures of which are all incorporated by reference in their entirety.

In certain embodiments, lactoferrin utilized in the present disclosure may be provided by an expanded bed absorption (“EBA”) process for isolating proteins from milk sources. EBA, also sometimes called stabilized fluid bed adsorption, is a process for isolating a milk protein, such as lactoferrin, from a milk source comprises establishing an expanded bed adsorption column comprising a particulate matrix, applying a milk source to the matrix, and eluting the lactoferrin from the matrix with an elution buffer comprising about 0.3 to about 2.0 M sodium chloride. Any mammalian milk source may be used in the present processes, although in particular embodiments, the milk source is a bovine milk source. The milk source comprises, in some embodiments, whole milk, reduced fat milk, skim milk, whey, casein, or mixtures thereof. In some embodiments, the process comprises the steps of establishing an expanded bed adsorption column comprising a particulate matrix, applying a milk source to the matrix, and eluting the lactoferrin from the matrix with about 0.3 to about 2.0M sodium chloride. In other embodiments, the lactoferrin is eluted with about 0.5 to about 1.0 M sodium chloride, while in further embodiments, the lactoferrin is eluted with about 0.7 to about 0.9 M sodium chloride.

The expanded bed adsorption column can be any known in the art, such as those described in U.S. Pat. Nos. 7,812,138, 6,620,326, and 6,977,046, the disclosures of which are hereby incorporated by reference herein. In some embodiments, a milk source is applied to the column in an expanded mode, and the elution is performed in either expanded or packed mode. In particular embodiments, the elution is performed in an expanded mode. For example, the expansion ratio in the expanded mode may be about 1 to about 3, or about 1.3 to about 1.7. EBA technology is further described in international published application nos. WO 92/00799, WO 02/18237, WO 97/17132, which are hereby incorporated by reference in their entireties.

The nutritional composition of the disclosure can also comprise DHA. DHA is present, in some embodiments, in an amount ranging from about 5 mg/100 Kcal to about 75 mg/100 Kcal, more preferably about 10 mg/100 Kcal to about 50 mg/100 Kcal. The DHA may be provided from any source of LCPUFAs. Other suitable LCPUFAs that may be present in certain embodiments of the present compositions include, but are not limited to, α-linoleic acid, γ-linoleic acid, linoleic acid, linolenic acid, eicosapentaenoic acid (EPA) and arachidonic acid (ARA).

In an embodiment, especially if the nutritional composition is an infant formula, the nutritional composition is supplemented with both DHA and ARA. In this embodiment, the weight ratio of ARA:DHA may be between about 1:3 and about 9:1. In a particular embodiment, the ratio of ARA:DHA is from about 1:2 to about 4:1.

The nutritional composition may be supplemented with oils comprising DHA and/or ARA using standard techniques known in the art. For example, DHA and ARA may be added to the composition by replacing an equivalent amount of an oil, such as high oleic sunflower oil, normally present in the composition. As another example, the oils comprising DHA and ARA may be added to the composition by replacing an equivalent amount of the rest of the overall fat blend normally present in the composition without DHA and ARA.

The source of DHA and ARA, when present, may be any source known in the art such as marine oil, fish oil, single cell oil, egg yolk lipid, and brain lipid. In some embodiments, the DHA and ARA are sourced from single cell Martek oils, DHASCO® and ARASCO®, or variations thereof. The DHA and ARA can be in natural form, provided that the remainder of the LCPUFA source does not result in any substantial deleterious effect on the infant. Alternatively, the DHA and ARA can be used in refined form.

In an embodiment, sources of DHA and ARA are single cell oils as taught in U.S. Pat. Nos. 5,374,567; 5,550,156; and 5,397,591, the disclosures of which are incorporated herein in their entirety by reference. However, the present disclosure is not limited to only such oils.

The nutritional composition may also comprise one or more prebiotics (also referred to as a prebiotic component) in certain embodiments. Prebiotics exert health benefits, which may include, but are not limited to, selective stimulation of the growth and/or activity of one or a limited number of beneficial gut bacteria, stimulation of the growth and/or activity of ingested probiotic microorganisms, selective reduction in gut pathogens, and favorable influence on gut short chain fatty acid profile. Such prebiotics may be naturally-occurring, synthetic, or developed through the genetic manipulation of organisms and/or plants, whether such new source is now known or developed later. Prebiotics useful in the present disclosure may include oligosaccharides, polysaccharides, and other prebiotics that comprise fructose, xylose, soya, galactose, glucose and mannose.

More specifically, prebiotics useful in the present disclosure may include polydextrose, polydextrose powder, lactose, lactulose, lactosucrose, raffinose, gluco-oligosaccharide, inulin, fructo-oligosaccharide, isomalto-oligosaccharide, soybean oligosaccharides, lactosucrose, xylo-oligosaccharide, chito-oligosaccharide, manno-oligosaccharide, aribino-oligosaccharide, siallyl-oligosaccharide, fuco-oligosaccharide, galacto-oligosaccharide and gentio-oligosaccharides.

In an embodiment, the total amount of prebiotics present in the nutritional composition may be from about 1.0 g/L to about 10.0 g/L of the composition. More preferably, the total amount of prebiotics present in the nutritional composition may be from about 2.0 g/L and about 8.0 g/L of the composition. In some embodiments, the total amount of prebiotics present in the nutritional composition may be from about 0.1 g/100 Kcal to about 1 g/100 Kcal. In certain embodiments, the total amount of prebiotics present in the nutritional composition may be from about 0.3 g/100 Kcal to about 0.7 g/100 Kcal. Moreover, the nutritional composition may comprise a prebiotic component comprising PDX. In some embodiments, the prebiotic component comprises at least 20% w/w PDX, GOS or a mixture thereof.

The amount of PDX in the nutritional composition may, in an embodiment, be within the range of from about 0.1 g/100 Kcal to about 1 g/100 Kcal. In another embodiment, the amount of polydextrose is within the range of from about 0.2 g/100 Kcal to about 0.6 g/100 Kcal. In still other embodiments, the amount of PDX in the nutritional composition may be from about 0.1 g/100 kcal to about 0.5 g/100 kcal.

The prebiotic component also comprises GOS in some embodiments. The amount of GOS in the nutritional composition may, in an embodiment, be from about 0.1 g/100 Kcal to about 1.0 g/100 Kcal. In another embodiment, the amount of GOS in the nutritional composition may be from about 0.2 g/100 Kcal to about 0.5 g/100 Kcal. In yet another embodiment, the amount GOS in the nutritional composition may be from about 0.1 g/100 kcal to about 0.5 g/100 kcal.

It is further believed that PDX and GOS have beneficial effect on brain development via the gut-brain-immune axis and therefore, when present, act synergistically to enhance brain development, and particularly, neuronal maturation.

The nutritional compositions of the disclosure may comprise at least one protein source. The protein source can be any used in the art, e.g., nonfat milk, whey protein, casein, soy protein, hydrolyzed protein, amino acids, and the like. Bovine milk protein sources useful in practicing the present disclosure include, but are not limited to, milk protein powders, milk protein concentrates, milk protein isolates, nonfat milk solids, nonfat milk, nonfat dry milk, whey protein, whey protein isolates, whey protein concentrates, sweet whey, acid whey, casein, acid casein, caseinate (e.g. sodium caseinate, sodium calcium caseinate, calcium caseinate) and any combinations thereof.

In some embodiments, the proteins of the nutritional composition are provided as intact proteins. In other embodiments, the proteins are provided as a combination of both intact proteins and hydrolyzed proteins. In certain embodiments, the proteins may be partially hydrolyzed or extensively hydrolyzed. In still other embodiments, the protein source comprises amino acids. In yet another embodiment, the protein source may be supplemented with glutamine-containing peptides.

In another embodiment, the protein component comprises extensively hydrolyzed protein. In still another embodiment, the protein component of the nutritional composition consists essentially of extensively hydrolyzed protein in order to minimize the occurrence of food allergy. In yet another embodiment, the protein source may be supplemented with glutamine-containing peptides.

Some people exhibit allergies or sensitivities to intact proteins, i.e. whole proteins, such as those in intact cow's milk protein or intact soy protein isolate-based formulas. Many of these people with protein allergies or sensitivities are able to tolerate hydrolyzed protein. Hydrolysate formulas (also referred to as semi-elemental formulas) contain protein that has been hydrolyzed or broken down into short peptide fragments and amino acids and as a result is more easily digested. In people with protein sensitivities or allergies, immune system associated allergies or sensitivities often result in cutaneous, respiratory or gastrointestinal symptoms such as vomiting and diarrhea. People who exhibit reactions to intact protein formulas often will not react to hydrolyzed protein formulas because their immune system does not recognize the hydrolyzed protein as the intact protein that causes their symptoms.

Accordingly, in some embodiments, the protein component of the nutritional composition comprises either partially or extensively hydrolyzed protein, such as protein from cow's milk. The hydrolyzed proteins may be treated with enzymes to break down some or most of the proteins that cause adverse symptoms with the goal of reducing allergic reactions, intolerance, and sensitization. Moreover, the proteins may be hydrolyzed by any method known in the art.

The terms “protein hydrolysates” or “hydrolyzed protein” are used interchangeably herein and refer to hydrolyzed proteins, wherein the degree of hydrolysis is may be from about 20% to about 80%, or from about 30% to about 80%, or even from about 40% to about 60%. The degree of hydrolysis is the extent to which peptide bonds are broken by a hydrolysis method. The degree of protein hydrolysis for purposes of characterizing the hydrolyzed protein component of the nutritional composition is easily determined by one of ordinary skill in the formulation arts by quantifying the amino nitrogen to total nitrogen ratio (AN/TN) of the protein component of the selected formulation. The amino nitrogen component is quantified by USP titration methods for determining amino nitrogen content, while the total nitrogen component is determined by the Kjeldahl method, all of which are well known methods to one of ordinary skill in the analytical chemistry art.

When a peptide bond in a protein is broken by enzymatic hydrolysis, one amino group is released for each peptide bond broken, causing an increase in amino nitrogen. It should be noted that even non-hydrolyzed protein would contain some exposed amino groups. Hydrolyzed proteins will also have a different molecular weight distribution than the non-hydrolyzed proteins from which they were formed. The functional and nutritional properties of hydrolyzed proteins can be affected by the different size peptides. A molecular weight profile is usually given by listing the percent by weight of particular ranges of molecular weight (in Daltons) fractions (e.g., 2,000 to 5,000 Daltons, greater than 5,000 Daltons).

As previously mentioned, persons who exhibit sensitivity to whole or intact proteins can benefit from consumption of nutritional formulas comprising hydrolyzed proteins. Such sensitive persons may especially benefit from the consumption of a hypoallergenic formula.

In some embodiments, the nutritional composition of the present disclosure is substantially free of intact proteins, other than the added lactoferrin. In this context, the term “substantially free” means that the preferred embodiments herein comprise sufficiently low concentrations of intact protein to thus render the formula hypoallergenic. The extent to which a nutritional composition in accordance with the disclosure is substantially free of intact proteins, and therefore hypoallergenic, is determined by the August 2000 Policy Statement of the American Academy of Pediatrics in which a hypoallergenic formula is defined as one which in appropriate clinical studies demonstrates that it does not provoke reactions in 90% of infants or children with confirmed cow's milk allergy with 95% confidence when given in prospective randomized, double-blind, placebo-controlled trials.

Another alternative for pediatric subjects, such as infants, that have food allergy and/or milk protein allergies is a protein-free nutritional composition based on amino acids. Amino acids are the basic structural building units of protein. Breaking the proteins down to their basic chemical structure by completely pre-digesting the proteins makes amino acid-based formulas the most hypoallergenic formulas available.

In a particular embodiment, the nutritional composition is protein-free and comprises free amino acids as a protein equivalent source (in addition to lactoferrin). In this embodiment, the amino acids may comprise, but are not limited to, histidine, isoleucine, leucine, lysine, methionine, cysteine, phenylalanine, tyrosine, threonine, tryptophan, valine, alanine, arginine, asparagine, aspartic acid, glutamic acid, glutamine, glycine, proline, serine, carnitine, taurine and mixtures thereof. In some embodiments, the amino acids may be branched chain amino acids. In other embodiments, small amino acid peptides may be included as the protein component of the nutritional composition. Such small amino acid peptides may be naturally occurring or synthesized. The amount of free amino acids in the nutritional composition may vary from about 1 to about 5 g/100 Kcal. In an embodiment, 100% of the free amino acids have a molecular weight of less than 500 Daltons. In this embodiment, the nutritional formulation may be hypoallergenic.

In a particular embodiment of the nutritional composition, the whey:casein ratio of the protein source is similar to that found in human breast milk. In an embodiment, the protein source comprises from about 40% to about 85% whey protein and from about 15% to about 60% casein.

In some embodiments, the nutritional composition comprises between about 1 g and about 7 g of a protein and/or protein equivalent source per 100 Kcal. In other embodiments, the nutritional composition comprises between about 3.5 g and about 4.5 g of protein or protein equivalent per 100 Kcal.

The nutritional composition of the present disclosure may comprise native or modified starches, such as, for example, waxy corn starch, waxy rice starch, corn starch, rice starch, potato starch, tapioca starch, wheat starch or any mixture thereof. Generally, common corn starch comprises about 25% amylose, while waxy corn starch is almost totally made up of amylopectin. Meanwhile, potato starch generally comprises about 20% amylose, rice starch comprises an amylose: amylopectin ratio of about 20:80, and waxy rice starch comprises only about 2% amylose. Further, tapioca starch generally comprises about 15% to about 18% amylose, and wheat starch has an amylose content of around 25%.

In some embodiments, the nutritional composition comprises gelatinized and/or pre-gelatinized waxy corn starch. In other embodiments, the nutritional composition comprises gelatinized and/or pre-gelatinized tapioca starch. Other gelatinized or pre-gelatinized starches, such as rice starch or potato starch may also be used.

Additionally, in some embodiments the nutritional compositions of the present disclosure comprise at least one source of pectin. The source of pectin may comprise any variety or grade of pectin known in the art. In some embodiments, the pectin has a degree of esterification of less than 50% and is classified as low methylated (“LM”) pectin. In some embodiments, the pectin has a degree of esterification of greater than or equal to 50% and is classified as high-ester or high methylated (“HM”) pectin. In still other embodiments, the pectin is very low (“VL”) pectin, which has a degree of esterification that is less than approximately 15%. Further, the nutritional composition of the present disclosure may comprise LM pectin, HM pectin, VL pectin, or any mixture thereof. The nutritional composition may include pectin that is soluble in water. And, as known in the art, the solubility and viscosity of a pectin solution are related to the molecular weight, degree of esterification, concentration of the pectin preparation and the pH and presence of counter ions.

Pectins for use herein typically have a peak molecular weight of 8,000 Daltons or greater. The pectins of the present disclosure have a preferred peak molecular weight of between 8,000 and about 500,000, more preferred is between about 10,000 and about 200,000 and most preferred is between about 15,000 and about 100,000 Daltons. In some embodiments, the pectin of the present disclosure may be hydrolyzed pectin. In certain embodiments, the nutritional composition comprises hydrolyzed pectin having a molecular weight less than that of intact or unmodified pectin. The hydrolyzed pectin of the present disclosure can be prepared by any means known in the art to reduce molecular weight. Examples of said means are chemical hydrolysis, enzymatic hydrolysis and mechanical shear. A preferred means of reducing the molecular weight is by alkaline or neutral hydrolysis at elevated temperature. In some embodiments, the nutritional composition comprises partially hydrolyzed pectin. In certain embodiments, the partially hydrolyzed pectin has a molecular weight that is less than that of intact or unmodified pectin but more than 3,300 Daltons.

In some embodiments, the nutritional composition comprises up to about 20% w/w of a mixture of starch and pectin. In some embodiments, the nutritional composition comprises up to about 19% starch and up to about 1% pectin. In other embodiments, the nutritional composition comprises about up to about 15% starch and up to about 5% pectin. In still other embodiments, the nutritional composition comprises up to about 18% starch and up to about 2% pectin. In some embodiments the nutritional composition comprises between about 0.05% w/w and about 20% w/w of a mixture of starch and pectin. Other embodiments include between about 0.05% and about 19% w/w starch and between about 0.05% and about 1% w/w pectin. Further, the nutritional composition may comprise between about 0.05% and about 15% w/w starch and between about 0.05% and about 5% w/w pectin.

In some embodiments, the nutritional composition comprises at least one additional carbohydrate, that is, a carbohydrate component provided in addition to the aforementioned starch component. Additional carbohydrate sources can be any used in the art, e.g., lactose, glucose, fructose, corn syrup solids, maltodextrins, sucrose, starch, rice syrup solids, and the like. The amount of the additional carbohydrate component in the nutritional composition typically can vary from between about 5 g and about 25 g/100 Kcal. In some embodiments, the amount of carbohydrate is between about 6 g and about 22 g/100 Kcal. In other embodiments, the amount of carbohydrate is between about 12 g and about 14 g/100 Kcal. In some embodiments, corn syrup solids are preferred. Moreover, hydrolyzed, partially hydrolyzed, and/or extensively hydrolyzed carbohydrates may be desirable for inclusion in the nutritional composition due to their easy digestibility. Specifically, hydrolyzed carbohydrates are less likely to contain allergenic epitopes.

Non-limiting examples of carbohydrate materials suitable for use herein include hydrolyzed or intact, naturally or chemically modified, starches sourced from corn, tapioca, rice or potato, in waxy or non-waxy forms. Non-limiting examples of suitable carbohydrates include various hydrolyzed starches characterized as hydrolyzed cornstarch, maltodextrin, maltose, corn syrup, dextrose, corn syrup solids, glucose, and various other glucose polymers and combinations thereof. Non-limiting examples of other suitable carbohydrates include those often referred to as sucrose, lactose, fructose, high fructose corn syrup, indigestible oligosaccharides such as fructooligosaccharides and combinations thereof.

Particular embodiments of the present compositions include lactose as a carbohydrate source. In one particular embodiment, the additional carbohydrate component of the nutritional composition is comprised of 100% lactose. In another embodiment, the additional carbohydrate component comprises between about 0% and 60% lactose. In another embodiment, the additional carbohydrate component comprises between about 15% and 55% lactose. In yet another embodiment, the additional carbohydrate component comprises between about 20% and 30% lactose. In these embodiments, the remaining source of carbohydrates may be any carbohydrate known in the art. In an embodiment, the carbohydrate component comprises about 25% lactose and about 75% corn syrup solids.

In some embodiments the nutritional composition comprises sialic acid. Sialic acids are a family of over 50 members of 9-carbon sugars, all of which are derivatives of neuraminic acid. The predominant sialic acid family found in humans is from the N-acetylneuraminic acid sub-family. Sialic acids are found in milk, such as bovine and caprine. In mammals, neuronal cell membranes have the highest concentration of sialic acid compared to other body cell membranes. Sialic acid residues are also components of gangliosides.

If included in the nutritional composition, sialic acid may be present in an amount from about 0.5 mg/100 Kcals to about 45 mg/100 Kcal. In some embodiments sialic acid may be present in an amount from about 5 mg/100 Kcals to about 30 mg/100 Kcals. In still other embodiments, sialic acid may be present in an amount from about 10 mg/100 Kcals to about 25 mg/100 Kcals.

The present nutritional composition may comprise a source of β-glucan. Glucans are polysaccharides, specifically polymers of glucose, which are naturally occurring and may be found in cell walls of bacteria, yeast, fungi, and plants. Beta glucans β-glucans) are themselves a diverse subset of glucose polymers, which are made up of chains of glucose monomers linked together via beta-type glycosidic bonds to form complex carbohydrates.

β-1,3-glucans are carbohydrate polymers purified from, for example, yeast, mushroom, bacteria, algae, or cereals. (Stone B A, Clarke A E. Chemistry and Biology of (1-3)-Beta-Glucans. London:Portland Press Ltd; 1993.) The chemical structure of β-1,3-glucan depends on the source of the β-1,3-glucan. Moreover, various physiochemical parameters, such as solubility, primary structure, molecular weight, and branching, play a role in biological activities of β-1,3-glucans. (Yadomae T., Structure and biological activities of fungal beta-1,3-glucans. Yakugaku Zasshi. 2000; 120:413-431.)

β-1,3-glucans are naturally occurring polysaccharides, with or without β-1,6-glucose side chains that are found in the cell walls of a variety of plants, yeasts, fungi and bacteria. β-1,3; 1,6-glucans are those containing glucose units with (1,3) links having side chains attached at the (1,6) position(s). β-1,3; 1,6 glucans are a heterogeneous group of glucose polymers that share structural commonalities, including a backbone of straight chain glucose units linked by a β-1,3 bond with β-1,6-linked glucose branches extending from this backbone. While this is the basic structure for the presently described class of β-glucans, some variations may exist. For example, certain yeast β-glucans have additional regions of β(1,3) branching extending from the β(1,6) branches, which add further complexity to their respective structures.

β-glucans derived from baker's yeast, Saccharomyces cerevisiae, are made up of chains of D-glucose molecules connected at the 1 and 3 positions, having side chains of glucose attached at the 1 and 6 positions. Yeast-derived β-glucan is an insoluble, fiber-like, complex sugar having the general structure of a linear chain of glucose units with a β-1,3 backbone interspersed with β-1,6 side chains that are generally 6-8 glucose units in length. More specifically, β-glucan derived from baker's yeast is poly-(1,6)-β-D-glucopyranosyl-(1,3)-β-D-glucopyranose.

Furthermore, β-glucans are well tolerated and do not produce or cause excess gas, abdominal distension, bloating or diarrhea, particularly in pediatric subjects. Addition of β-glucan to a nutritional composition for a pediatric subject, such as an infant formula, a growing-up milk or another children's nutritional product, will improve the subject's immune response by increasing resistance against invading pathogens and therefore maintaining or improving overall health.

The nutritional composition of the present disclosure comprises β-glucan. In some embodiments, the β-glucan is β-1,3; 1,6-glucan. In some embodiments, the β-1,3; 1,6-glucan is derived from baker's yeast. The nutritional composition may comprise whole glucan particle β-glucan, particulate β-glucan, PGG-glucan (poly-1,6-β-D-glucopyranosyl-1,3-1-D-glucopyranose) or any mixture thereof.

In some embodiments, the amount of β-glucan present in the composition is at between about 0.010 and about 0.080 g per 100 g of composition. In other embodiments, the nutritional composition comprises between about 10 and about 30 mg β-glucan per serving. In another embodiment, the nutritional composition comprises between about 5 and about 30 mg β-glucan per 8 fl. oz. (236.6 mL) serving. In other embodiments, the nutritional composition comprises an amount of β-glucan sufficient to provide between about 15 mg and about 90 mg β-glucan per day. The nutritional composition may be delivered in multiple doses to reach a target amount of β-glucan delivered to the subject throughout the day. In some embodiments, the amount of β-glucan in the nutritional composition is between about 3 mg and about 17 mg per 100 Kcal. In another embodiment the amount of β-glucan is between about 6 mg and about 17 mg per 100 Kcal.

It has been found that nutritional supplementation of inositol represents a feasible and effective approach to promote oligodendrocyte survival and proliferation in a dose dependent manner, resulting in a consistent increase in the number of oligodendrocyte precursor cells. Nutritional supplementation with inositol provides benefits for enhanced developmental myelination by which it translates into a fundamental benefit for brain development. Given the importance of functional myelination, nutritional supplementation of inositol is beneficial to pediatric subjects by enhancing brain development and health. Moreover, the sweet taste of inositol provides further advantages in terms of palatability to pediatric consumers.

As such, in certain embodiments, inositol is present in the nutritional compositions of the present disclosure at a level of at least about 4 mg/100 Kcal; in other embodiments, inositol should be present at a level of no greater than about 70 mg/100 Kcal. In still other embodiments, the nutritional composition comprises inositol at a level of about 5 mg/100 Kcal to about 65 mg/100 Kcal. In a further embodiment, inositol is present in the nutritional composition at a level of about 7 mg/100 Kcal to about 50 mg/100 Kcal. Moreover, inositol can be present as exogenous inositol or inherent inositol. In embodiments, a major fraction of the inositol (i.e., at least 40%) is exogenous inositol. In certain embodiments, the ratio of exogenous to inherent inositol is at least 50:50; in other embodiments, the ratio of exogenous to inherent inositol is at least 60:40.

One or more vitamins and/or minerals may also be added in to the nutritional composition in amounts sufficient to supply the daily nutritional requirements of a subject. It is to be understood by one of ordinary skill in the art that vitamin and mineral requirements will vary, for example, based on the age of the child. For instance, an infant may have different vitamin and mineral requirements than a child between the ages of one and thirteen years. Thus, the embodiments are not intended to limit the nutritional composition to a particular age group but, rather, to provide a range of acceptable vitamin and mineral components.

The nutritional composition may optionally include, but is not limited to, one or more of the following vitamins or derivations thereof: vitamin B₁ (thiamin, thiamin pyrophosphate, TPP, thiamin triphosphate, TTP, thiamin hydrochloride, thiamin mononitrate), vitamin B₂ (riboflavin, flavin mononucleotide, FMN, flavin adenine dinucleotide, FAD, lactoflavin, ovoflavin), vitamin B₃ (niacin, nicotinic acid, nicotinamide, niacinamide, nicotinamide adenine dinucleotide, NAD, nicotinic acid mononucleotide, NicMN, pyridine-β-carboxylic acid), vitamin B₃-precursor tryptophan, vitamin B₆ (pyridoxine, pyridoxal, pyridoxamine, pyridoxine hydrochloride), pantothenic acid (pantothenate, panthenol), folate (folic acid, folacin, pteroylglutamic acid), vitamin B₁₂ (cobalamin, methylcobalamin, deoxyadenosylcobalamin, cyanocobalamin, hydroxycobalamin, adenosylcobalamin), biotin, vitamin C (ascorbic acid), vitamin A (retinol, retinyl acetate, retinyl palmitate, retinyl esters with other long-chain fatty acids, retinal, retinoic acid, retinol esters), vitamin D (calciferol, cholecalciferol, vitamin D₃, 1,25,-dihydroxyvitamin D), vitamin E (α-tocopherol, α-tocopherol acetate, α-tocopherol succinate, α-tocopherol nicotinate, α-tocopherol), vitamin K (vitamin K₁, phylloquinone, naphthoquinone, vitamin K₂, menaquinone-7, vitamin K₃, menaquinone-4, menadione, menaquinone-8, menaquinone-8H, menaquinone-9, menaquinone-9H, menaquinone-10, menaquinone-11, menaquinone-12, menaquinone-13), choline, inositol, β-carotene and any combinations thereof.

Further, the nutritional composition may optionally include, but is not limited to, one or more of the following minerals or derivations thereof: boron, calcium, calcium acetate, calcium gluconate, calcium chloride, calcium lactate, calcium phosphate, calcium sulfate, chloride, chromium, chromium chloride, chromium picolonate, copper, copper sulfate, copper gluconate, cupric sulfate, fluoride, iron, carbonyl iron, ferric iron, ferrous fumarate, ferric orthophosphate, iron trituration, polysaccharide iron, iodide, iodine, magnesium, magnesium carbonate, magnesium hydroxide, magnesium oxide, magnesium stearate, magnesium sulfate, manganese, molybdenum, phosphorus, potassium, potassium phosphate, potassium iodide, potassium chloride, potassium acetate, selenium, sulfur, sodium, docusate sodium, sodium chloride, sodium selenate, sodium molybdate, zinc, zinc oxide, zinc sulfate and mixtures thereof. Non-limiting exemplary derivatives of mineral compounds include salts, alkaline salts, esters and chelates of any mineral compound.

The minerals can be added to nutritional compositions in the form of salts such as calcium phosphate, calcium glycerol phosphate, sodium citrate, potassium chloride, potassium phosphate, magnesium phosphate, ferrous sulfate, zinc sulfate, cupric sulfate, manganese sulfate, and sodium selenite. Additional vitamins and minerals can be added as known within the art.

In an embodiment, the nutritional composition may comprise between about 10 and about 50% of the maximum dietary recommendation for any given country, or between about 10 and about 50% of the average dietary recommendation for a group of countries, per serving of vitamins A, C, and E, zinc, iron, iodine, selenium, and choline. In another embodiment, the children's nutritional composition may supply about 10-30% of the maximum dietary recommendation for any given country, or about 10-30% of the average dietary recommendation for a group of countries, per serving of B-vitamins. In yet another embodiment, the levels of vitamin D, calcium, magnesium, phosphorus, and potassium in the children's nutritional product may correspond with the average levels found in milk. In other embodiments, other nutrients in the children's nutritional composition may be present at about 20% of the maximum dietary recommendation for any given country, or about 20% of the average dietary recommendation for a group of countries, per serving.

The nutritional compositions of the present disclosure may optionally include one or more of the following flavoring agents, including, but not limited to, flavored extracts, volatile oils, cocoa or chocolate flavorings, peanut butter flavoring, cookie crumbs, vanilla or any commercially available flavoring. Examples of useful flavorings include, but are not limited to, pure anise extract, imitation banana extract, imitation cherry extract, chocolate extract, pure lemon extract, pure orange extract, pure peppermint extract, honey, imitation pineapple extract, imitation rum extract, imitation strawberry extract, or vanilla extract; or volatile oils, such as balm oil, bay oil, bergamot oil, cedarwood oil, cherry oil, cinnamon oil, clove oil, or peppermint oil; peanut butter, chocolate flavoring, vanilla cookie crumb, butterscotch, toffee, and mixtures thereof. The amounts of flavoring agent can vary greatly depending upon the flavoring agent used. The type and amount of flavoring agent can be selected as is known in the art.

The nutritional compositions of the present disclosure may optionally include one or more emulsifiers that may be added for stability of the final product. Examples of suitable emulsifiers include, but are not limited to, lecithin (e.g., from egg or soy), alpha lactalbumin and/or mono- and di-glycerides, and mixtures thereof. Other emulsifiers are readily apparent to the skilled artisan and selection of suitable emulsifier(s) will depend, in part, upon the formulation and final product.

The nutritional compositions of the present disclosure may optionally include one or more preservatives that may also be added to extend product shelf life. Suitable preservatives include, but are not limited to, potassium sorbate, sodium sorbate, potassium benzoate, sodium benzoate, calcium disodium EDTA, and mixtures thereof.

The nutritional compositions of the present disclosure may optionally include one or more stabilizers. Suitable stabilizers for use in practicing the nutritional composition of the present disclosure include, but are not limited to, gum arabic, gum ghatti, gum karaya, gum tragacanth, agar, furcellaran, guar gum, gellan gum, locust bean gum, pectin, low methoxyl pectin, gelatin, microcrystalline cellulose, CMC (sodium carboxymethylcellulose), methylcellulose hydroxypropyl methyl cellulose, hydroxypropyl cellulose, DATEM (diacetyl tartaric acid esters of mono- and diglycerides), dextran, carrageenans, and mixtures thereof.

The disclosed nutritional composition(s) may be provided in any form known in the art, such as a powder, a gel, a suspension, a paste, a solid, a liquid, a liquid concentrate, a reconstitutable powdered milk substitute or a ready-to-use product. The nutritional composition may, in certain embodiments, comprise a nutritional supplement, children's nutritional product, infant formula, human milk fortifier, growing-up milk or any other nutritional composition designed for an infant or a pediatric subject. Nutritional compositions of the present disclosure include, for example, orally-ingestible, health-promoting substances including, for example, foods, beverages, tablets, capsules and powders. Moreover, the nutritional composition of the present disclosure may be standardized to a specific caloric content, it may be provided as a ready-to-use product, or it may be provided in a concentrated form. In some embodiments, the nutritional composition is in powder form with a particle size in the range of 5 μm to 1500 μm, more preferably in the range of 10 μm to 300 μm.

If the nutritional composition is in the form of a ready-to-use product, the osmolality of the nutritional composition may be between about 100 and about 1100 mOsm/kg water, more typically about 200 to about 700 mOsm/kg water.

Suitable fat or lipid sources for the nutritional composition of the present disclosure may be any known or used in the art, including but not limited to, animal sources, e.g., milk fat, butter, butter fat, egg yolk lipid; marine sources, such as fish oils, marine oils, single cell oils; vegetable and plant oils, such as corn oil, canola oil, sunflower oil, soybean oil, palm olein oil, coconut oil, high oleic sunflower oil, evening primrose oil, rapeseed oil, olive oil, flaxseed (linseed) oil, cottonseed oil, high oleic safflower oil, palm stearin, palm kernel oil, wheat germ oil; medium chain triglyceride oils and emulsions and esters of fatty acids; and any combinations thereof.

The nutritional compositions of the disclosure may provide minimal, partial or total nutritional support. The compositions may be nutritional supplements or meal replacements. The compositions may, but need not, be nutritionally complete. In an embodiment, the nutritional composition of the disclosure is nutritionally complete and comprises suitable types and amounts of lipid, carbohydrate, protein, vitamins and minerals. The amount of lipid or fat typically can vary from about 1 to about 7 g/l 00 Kcal. The amount of protein typically can vary from about 1 to about 7 g/100 Kcal. The amount of carbohydrate typically can vary from about 6 to about 22 g/100 Kcal.

In an embodiment, the nutritional composition(s) of the present disclosure comprises an effective amount of choline. Choline is a nutrient that is essential for normal function of cells. It is a precursor for membrane phospholipids, and it accelerates the synthesis and release of acetylcholine, a neurotransmitter involved in memory storage. Moreover, though not wishing to be bound by this or any other theory, it is believed that dietary choline and docosahexaenoic acid (DHA) act synergistically to promote the biosynthesis of phosphatidylcholine and thus help promote synaptogenesis in human subjects. Additionally, choline and DHA may exhibit the synergistic effect of promoting dendritic spine formation, which is important in the maintenance of established synaptic connections. In some embodiments, the nutritional composition(s) of the present disclosure includes an effective amount of choline, which is about 20 mg choline per 8 fl. oz. (236.6 mL) serving to about 100 mg per 8 fl. oz. (236.6 mL) serving.

Moreover, in some embodiments, the nutritional composition is nutritionally complete, comprising suitable types and amounts of lipids, carbohydrates, proteins, vitamins and minerals to be a subject's sole source of nutrition. Indeed, the nutritional composition may optionally include any number of proteins, peptides, amino acids, fatty acids, probiotics and/or their metabolic by-products, prebiotics, carbohydrates and any other nutrient or other compound that may provide many nutritional and physiological benefits to a subject. Further, the nutritional composition of the present disclosure may comprise flavors, flavor enhancers, sweeteners, pigments, vitamins, minerals, therapeutic ingredients, functional food ingredients, food ingredients, processing ingredients or combinations thereof.

The LGG soluble mediator preparation or nutritional composition comprising the LGG soluble mediator preparation may be expelled directly into a subject's intestinal tract. In some embodiments, the nutritional composition is expelled directly into the gut. In some embodiments, the composition may be formulated to be consumed or administered enterally under the supervision of a physician and may be intended for the specific dietary management of a disease or condition, such as celiac disease and/or food allergy, for which distinctive nutritional requirements, based on recognized scientific principles, are established by medical evaluation

The nutritional composition of the present disclosure is not limited to compositions comprising nutrients specifically listed herein. Any nutrients may be delivered as part of the composition for the purpose of meeting nutritional needs and/or in order to optimize the nutritional status in a subject.

In some embodiments, the nutritional composition may be delivered to an infant from birth until a time that matches full-term gestation. In some embodiments, the nutritional composition may be delivered to an infant until at least about three months corrected age. In yet another embodiment, the nutritional composition may be delivered to an infant from birth until at least about six months corrected age. In yet another embodiment, the nutritional composition may be delivered to an infant from birth until at least about one year corrected age.

In certain embodiments, the nutritional composition is delivered to a subject as long as is necessary to achieve a reduction in severity of the viral infection, bacterial infection, and/or viral and bacterial co-infection, and/or to achieve a reduction in inflammation (e.g., lung inflammation) due to a viral infection, bacterial infection, and/or viral and bacterial co-infection.

The nutritional composition of the present disclosure may be standardized to a specific caloric content, it may be provided as a ready-to-use product, or it may be provided in a concentrated form.

In some embodiments, the nutritional composition of the present disclosure is a growing-up milk. Growing-up milks are fortified milk-based beverages intended for children over 1 year of age (typically from 1-3 years of age, from 4-6 years of age or from 1-6 years of age). They are not medical foods and are not intended as a meal replacement or a supplement to address a particular nutritional deficiency. Instead, growing-up milks are designed with the intent to serve as a complement to a diverse diet to provide additional insurance that a child achieves continual, daily intake of all essential vitamins and minerals, macronutrients plus additional functional dietary components, such as non-essential nutrients that have purported health-promoting properties.

The exact composition of a nutritional composition according to the present disclosure can vary from market-to-market, depending on local regulations and dietary intake information of the population of interest. In some embodiments, nutritional compositions according to the disclosure consist of a milk protein source, such as whole or skim milk, plus added sugar and sweeteners to achieve desired sensory properties, and added vitamins and minerals. The fat composition is typically derived from the milk raw materials. Total protein can be targeted to match that of human milk, cow milk or a lower value. Total carbohydrate is usually targeted to provide as little added sugar, such as sucrose or fructose, as possible to achieve an acceptable taste. Typically, Vitamin A, calcium and Vitamin D are added at levels to match the nutrient contribution of regional cow milk. Otherwise, in some embodiments, vitamins and minerals can be added at levels that provide approximately 20% of the dietary reference intake (DRI) or 20% of the Daily Value (DV) per serving. Moreover, nutrient values can vary between markets depending on the identified nutritional needs of the intended population, raw material contributions and regional regulations.

In certain embodiments, the nutritional composition is hypoallergenic. In other embodiments, the nutritional composition is kosher. In still further embodiments, the nutritional composition is a non-genetically modified product. In an embodiment, the nutritional formulation is sucrose-free. The nutritional composition may also be lactose-free. In other embodiments, the nutritional composition does not contain any medium-chain triglyceride oil. In some embodiments, no carrageenan is present in the composition. In other embodiments, the nutritional composition is free of all gums.

In some embodiments, the disclosure is directed to a staged nutritional feeding regimen for a pediatric subject, such as an infant or child, which includes a plurality of different nutritional compositions according to the present disclosure. Each nutritional composition comprises a hydrolyzed protein, at least one pre-gelatinized starch, and at least one pectin. In certain embodiments, the nutritional compositions of the feeding regimen may also include a source of long chain polyunsaturated fatty acid, at least one prebiotic, an iron source, a source of β-glucan, vitamins or minerals, lutein, zeaxanthin, or any other ingredient described hereinabove. The nutritional compositions described herein may be administered once per day or via several administrations throughout the course of a day.

Examples are provided to illustrate some embodiments of the nutritional composition of the present disclosure but should not be interpreted as any limitation thereon. Other embodiments within the scope of the claims herein will be apparent to one skilled in the art from the consideration of the specification or practice of the nutritional composition or methods disclosed herein. It is intended that the specification, together with the example, be considered to be exemplary only, with the scope and spirit of the disclosure being indicated by the claims which follow the example.

### EXAMPLES

### Example 1

Two LGG soluble mediator preparations (LEGa and LEGb) or unconditioned bacterial culture medium (as a reference) were orally administered to the mice on alternate days for 24 days post weaning. LEGa refers to soluble mediator desalted by column chromatography and LEGb refers to soluble mediator desalted by ultrafiltration. The unconditioned bacterial culture medium was processed in the same way as was LEGb. Each administration contained an average of 5×10⁸ colony forming units (CFU) equivalent/animal (or a corresponding amount of unconditioned culture medium).

At day 20 post weaning, animals were nasally infected with influenza virus, followed 5 days later with sub-lethal S. pneumonia bacterial infection. The control groups (both infected and non-infected) received water without any supplementation. Body weight was monitored at 0, 24, 48, and 72 hours relative to the time point of the S. pneumonia infection. Nose lavage and lung homogenates were collected at 24 h and 72 h post infection for bacterial counts and immune marker measurements.

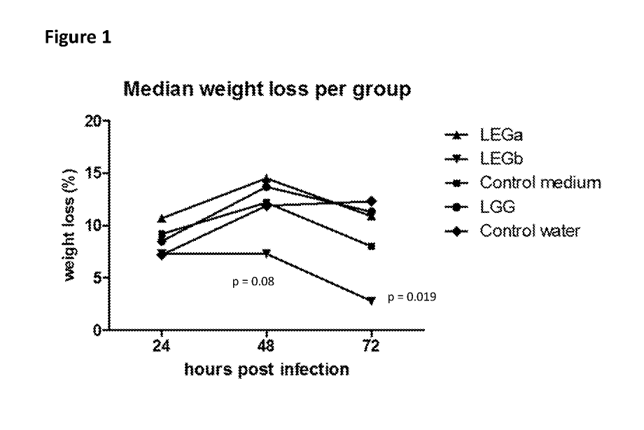

The majority of animals displayed rapid weight loss (up to 16%) after the influenza virus and S. pneumonia co-infection. However, the early (24 h) dampening effect of LEGb supplementation corresponded with less weight loss (p=0.0189) at 72 h (FIG. 1).

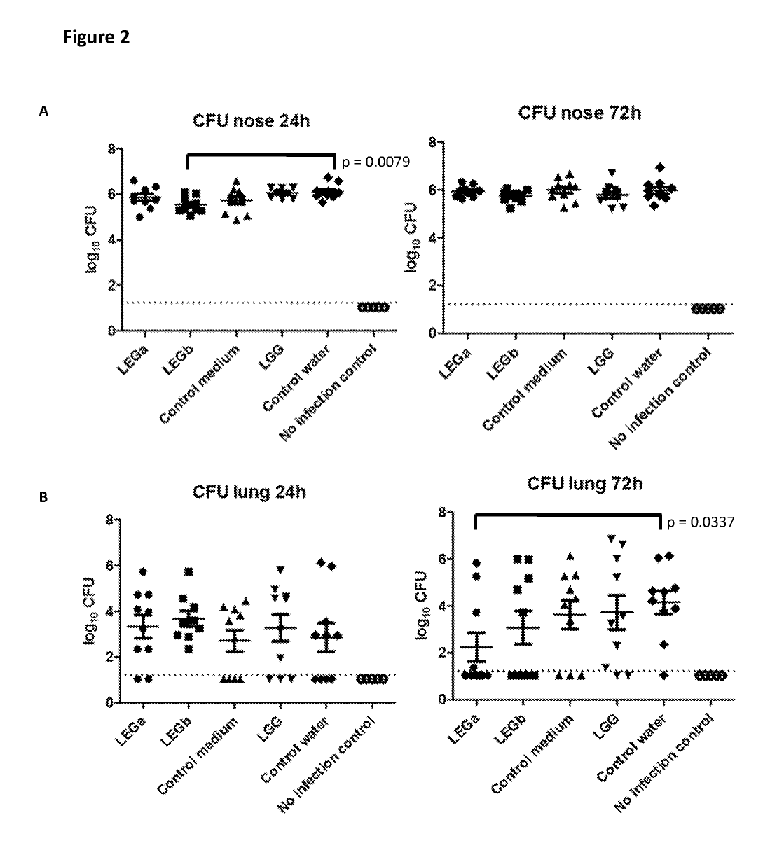

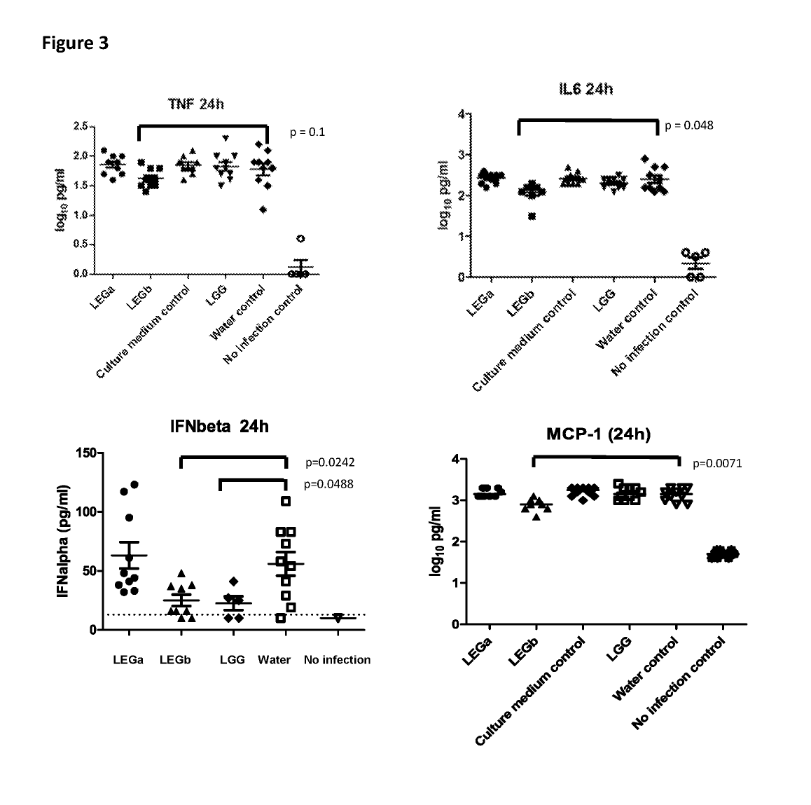

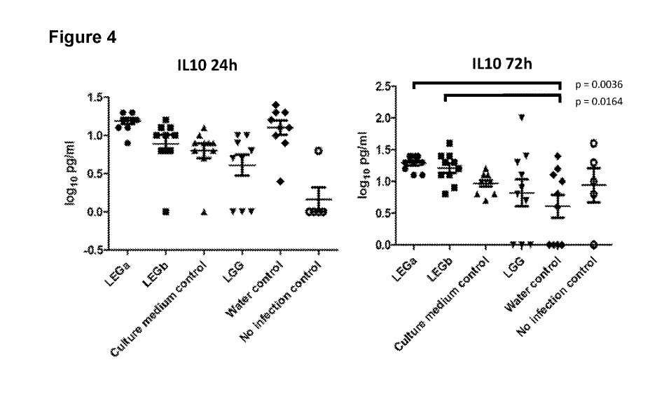

The results further showed that mice supplemented with LGG soluble mediator preparation B (LEGb) had lower pneumococcal counts in the nose 24 h after bacterial infection (p=0.0079) (FIG. 2A). At the same time point, these animals showed reduced amounts of pro-inflammatory cytokines IL-6 (p=0.048) and IFNβ (p-0.024) and chemoattractant protein MCP-1 (p=0.0071) in the lung (FIG. 3). These results suggest an anti-inflammatory effect of LEGb early after co-infection. Furthermore, the dampening effect of LEGb was sustained to 72 h, indicated by higher amounts of the anti-inflammatory cytokine IL-10 compared to infected control animals (FIG. 4).

These observations suggest that an early immune modulation in the lung mediated by oral administration of LEGb resulted in less severe symptoms of the respiratory tract co-infection.

Importantly, animals supplemented with unconditioned medium responded similarly to the infected control group receiving water, indicating that the beneficial effects of LEGb are indeed caused by active components secreted by LGG and not by any factors from the culture medium as such.

Although preferred embodiments of the disclosure have been described using specific terms, devices, and methods, such description is for illustrative purposes only. The words used are words of description rather than of limitation. It is to be understood that changes and variations may be made by those of ordinary skill in the art without departing from the spirit or the scope of the present disclosure, which is set forth in the following claims. In addition, it should be understood that aspects of the various embodiments may be interchanged in whole or in part. Therefore, the spirit and scope of the appended claims should not be limited to the description of the preferred versions contained therein.

What is claimed is:

1. A method for reducing the risk of developing a viral infection, bacterial infection, and/or viral and bacterial co-infection or reducing the severity of a viral infection, bacterial infection, and/or viral and bacterial co-infection in a subject in need thereof, comprising:
administering to the subject a nutritional composition comprising an effective amount of a soluble mediator preparation from a late-exponential growth phase of a probiotic culture.

2. The method of claim 1, wherein the probiotic is Lactobacillus rhamnosus GG (LGG).

3. The method of claim 2, wherein the subject is infected with a bacteria selected from the group consisting of Streptococcus pneumoniae, Haemophilus influenzae; Chlamydophila pneumoniae, Mycoplasma pneumoniae, Staphylococcus aureus, Moraxella catarrhalis, Legionella pneumophila, Gram-negative bacilli, Mycobacterium tuberculosis, Bordetella pertussis, Bordetella bronchiseptica, Streptococcus pyogenes, and Pseudomonas aeruginosa.

4. The method of claim 2, wherein the subject is infected with a virus selected from the group consisting of influenza A, influenza B, parainfluenza, human rhinovirus, adenovirus, respiratory syncytial virus (RSV), hantavirus, human metapneumovirus, Coronavirus, and nontypeable H. influenza (NTHi).

5. The method of claim 2, wherein the subject is a pediatric subject.

6. The method of claim 5, wherein the pediatric subject is a child, an infant, or a preterm infant.

7. The method of claim 2, wherein the soluble mediator preparation is produced by (a) subjecting LGG to cultivation in a suitable culture medium; (b) harvesting a culture supernatant at a late exponential growth phase of the cultivation step; (c) optionally removing low molecular weight constituents from the supernatant so as to retain molecular weight constituents above 5 or 6 kDa; (d) removing any remaining cells by 0.2 μm sterile filtration to provide the soluble mediator preparation; (e) removing liquid contents from the soluble mediator preparation.

8. The method of claim 7, wherein step (b) further comprises removal of bacterial cells by sterile filtration.

9.-11. (canceled)

12. The method of claim 2, wherein the nutritional composition is pediatric nutritional composition.

13. The method of claim 2, wherein the effective amount is equivalent to about 1×10⁴ to about 1×10¹² cfu probiotic bacteria per kg body weight per day.

14. A method for reducing inflammation in a subject with a viral infection, bacterial infection, and/or viral and bacterial co-infection, the method comprising:
administering to the subject a nutritional composition comprising an effective amount of a soluble mediator preparation from a late-exponential growth phase of a probiotic culture.

15. The method of claim 14, wherein the probiotic is Lactobacillus rhamnosus GG (LGG).

16. The method of claim 15, wherein the subject is infected with a bacteria selected from the group consisting of Streptococcus pneumoniae, Haemophilus influenzae; Chlamydophila pneumoniae, Mycoplasma pneumoniae, Staphylococcus aureus, Moraxella catarrhalis, Legionella pneumophila and Gram-negative bacilli.

17. The method of claim 15, wherein the subject is infected with a virus selected from the group consisting of influenza A, influenza B, parainfluenza, human rhinovirus, adenovirus, respiratory syncytial virus (RSV), hantavirus, and human meta pneumovirus.

18. The method of claim 15, wherein the subject is a pediatric subject.

19. The method of claim 18, wherein the pediatric subject is a child, infant, or preterm infant.

20. The method of claim 15, wherein the soluble mediator preparation is produced by (a) subjecting LGG to cultivation in a suitable culture medium; (b) harvesting the culture supernatant at a late exponential growth phase of the cultivation step; (c) optionally removing low molecular weight constituents from the supernatant so as to retain molecular weight constituents above 5 or 6 kDa; (d) removing any remaining cells by 0.2 μm sterile filtration; (e) removing liquid contents from the soluble mediator preparation.

21.-23. (canceled)

24. The method of claim 15, wherein the nutritional composition is pediatric nutritional composition.

25. The method of claim 15, wherein the effective amount is equivalent to about 1×10⁴ to about 1×10¹² cell equivalents of live probiotic bacteria per kg body weight per day.

26. The method of claim 15, wherein the method results in
(a) a reduction in a pro-inflammatory cytokine selected from the group consisting of IL-6, IFNβ, and TNFα, or
(b) a reduction in neutrophil or macrophage recruitment, or
(c) a reduction in chemoattractant protein MCP-1 or
(d) an increase in IL-10.
